(19) United States
(12) Patent Application Publication
(10) Pub. No.: US 2003/0099635 A1
(43) Pub. Date: May 29, 2003

(54) Use of oral gammaglobulin for the treatment of immune-mediated diseases

(75) Inventors: Leon E. Barstow, Tucson, AZ (US); Richard Weisbart, Los Angeles, CA (US); James A. Ostrem, Tucson, AZ (US); F. Javier Enriquez, Tucson, AZ (US)

Correspondence Address: FULBRIGHT & JAWORSKI, LLP, 1301 MCKINNEY, SUITE 5100, HOUSTON, TX, 77010-3095, US

(73) Assignee: Protein Therapeutics, Inc.

(21) Appl. No.: 10/264,564

(22) Filed: Oct. 4, 2002

Related U.S. Application Data
(60) Provisional application No. 60/327,043, filed on Oct. 4, 2001.
(60) Provisional application No. 60/380,960, filed on May 16, 2002.

(51) Int. Cl. A61K 39/395

(52) U.S. Cl. 424/130.1

(57) ABSTRACT

The present invention relates to the method of treatment for immune-mediated neurodegenerative diseases using alimentary administration, for example oral administration, of immunoglobulin. More particular, autistic spectrum disorder is treated using oral administration of immunoglobulin.

This application claims priority to U.S. Provisional Application No. 60/327,043, which was filed on Oct. 4, 2001 and U.S. Provisional Application No. 60/380,960, which was filed on May 16, 2002.

### BACKGROUND OF THE INVENTION

A. Field of Invention

The present invention relates to the fields of neurology and immunology. More particularly, the present invention relates to a method of treating immune-mediated neurodegenerative disease by administering to a subject via an alimentary route an immunoglobulin composition. In particular, the neurodegenerative disease is autistic spectrum disorder.

B. Related Art

1. Immune-mediated Diseases

Immune-mediated diseases are chronic inflammatory diseases perpetuated by antibodies and cellular immunity. The immune response damages healthy organs either inadvertently as a result of attacking foreign substances that have entered the body, or by attacking self tissues that happen to resemble foreign substances, a process called autoimmunity. These diseases include many forms of arthritis (e.g., rheumatoid arthritis and psoriatic arthritis), inflammatory bowel diseases (e.g., ulcerative colitis and Crohn's disease), endocrinopathies (e.g., type 1 diabetes and Graves disease), neurodegenerative diseases (e.g., multiple sclerosis, autistic spectrum disorder, Alzheimer's disease, amyotrophic lateral sclerosis (ALS), Parkinson's disease, Huntington's Disease, Guillain-Barre syndrome, myasthenia gravis, and chronic idiopathic demyelinating disease (CID)), and vascular diseases (e.g., autoimmune hearing loss, systemic vasculitis, and atherosclerosis). These diseases are common and have a major socioeconomic impact.

Currently, the primary focus of therapy is to suppress immunity and inhibit inflammation. However, immune reactivity and inflammation are critical for host defense against microbial pathogens and cancer. Therefore, the major drawback of current therapies is the predisposition to infection and cancer. Although many of the current therapies are effective, their prolonged use is often precluded by toxicity.

Immune-mediated diseases are complex and multifactorial. Recent research indicates that immune-mediated diseases require both a genetic predisposition and an environmental trigger. In many cases microbes have been implicated as a primary stimulus, and the gastrointestinal tract is a common source of these microbes. It is now recognized that some microbes implicated in autoimmune diseases may be ubiquitous, and the development of disease is determined by the genetic makeup of susceptible individuals.

A major clue to the cause of autoimmunity is its association with immunodeficiency. The most common immunodeficiency is the absence and/or decrease of IgA antibodies. IgA antibodies are secreted into the gastrointestinal tract as an important host defense mechanism. These antibodies protect us from numerous bacterial toxins by neutralizing them. Even in the presence of IgA, immunodeficiency may occur through mechanisms as subtle as a fortuitous similarity in appearance between self and microbe.

2. Autistic Spectrum Disorder

Autistic spectrum disorder (ASD) is a complex developmental disability. The disability affects social interaction and communication skills. Children and adults with autism have difficulties with verbal and non-verbal communication, social interactions and leisure activities.

Many ASD children appear intolerant to common dietary protein antigens (Ag), suffering from a variety of gastrointestinal (GI) signs and symptoms including diarrhea, constipation, colic, gastroesophageal reflux (GER), and GI discomfort. Parents of ASD children frequently report improvement of their GI symptoms as well as their aberrant behavior following implementation of an elimination diet (Berney 2001).

The GI mucosa is important for inducing immunological tolerance against numerous dietary proteins. However, tolerance induction in the peripheral lymphoid organs is regulated by sophisticated and complex mechanisms (Krause et al., 2000). Dysregulated activation of immune reactivity in the GI mucosa can lead to activation of autoreactive T cells and autoantibody production. It is well known that microbial infection in the gut often precipitates disease exacerbation in inflammatory bowel diseases (IBD) and other systemic autoimmune disorders. The gut associated mucosal immune system may be crucial to maintain immunological tolerance.

Pooled gammaglobulin obtained from thousands of adult donors are known to exert anti-inflammatory actions in various autoimmune disorders when given intravenously in a large amount without inducing immunosuppression. Intravenous gammaglobulin (IVIG) exerts various beneficial effects to attenuate autoimmune conditions via numerous direct and indirect (immunomodulatory) mechanisms of action (Kazatchkine and Kaveri, 2001). It has been reported that IVIG improved clinical features of autism in a small subset of ASD patients (Gupta et al., 1996), although the mechanism of its action was unknown.

Although IVIG administration has resulted in some improvements, IVIG is expensive and requires a prolonged infusion hours every 3-4 weeks. Thus, the development of a safer and more convenient therapy is necessary for the treatment of ASD and other immune-mediated neurodegenerative diseases.

### BRIEF SUMMARY OF THE INVENTION

The present invention is directed to a method for treating immune-mediated diseases. The method of treatment involves administration of immunoglobulin via an alimentary route, for example, oral, rectal, sublingual or buccal. In specific embodiments, the immune-mediated disease is a neurodegenerative disease, for example autistic spectrum disorder. Yet further, the present invention also includes treatment of GI manifestations associated with neurodegenerative disorders.

One embodiment of the present invention is a method of treating an immune-mediated neurodegenerative disease in a subject comprising the step of administering to the subject via an alimentary route an immunoglobulin composition in an amount sufficient to provide an improvement in the neurodegenerative disease in the subject. Specific examples of alimentary routes include, but are not limited to oral, buccal, rectal and sublingual. More particularly, the alimentary route is an oral route. In further embodiments, the immunoglobulin composition is administered in conjunction with an antacid. Yet further, the antacid is administered prior to or simultaneously with the immunoglobulin composition.

In specific embodiments, the neurodegenerative disease is selected from the group consisting of multiple sclerosis, autism and Alzheimer's disease. Yet further, the immunoglobulin composition comprises human immunoglobulin that can be dispersed in a pharmaceutically acceptable carrier. More particularly, the human immunoglobulin is human immunoglobulin G, immunoglobulin A or a combination thereof

Another embodiment is a method of treating an autistic spectrum disorder in a subject comprising the step of administering to the subject via an alimentary route an immunoglobulin composition in an amount sufficient to provide an improvement in the autistic spectrum disorder in the subject.

Yet further, another embodiment is a method of treating an immune-mediated disease comprising the step of supplementing a mucosal immune system. The immune-mediated disease is an autistic spectrum disorder. More particularly, supplementing the mucosal immune system comprises increasing the amount of immunoglobulin G, immunoglobulin A or a combination thereof in the gastrointestinal tract. Yet further, in specific embodiments, the immunoglobulin G, immunoglobulin A or a combination thereof is administered to the subject via an alimentary route. In preferred embodiments, the alimentary route is an oral route.

Still further, another embodiments is a method of enhancing a mucosal immune response in the gastrointestinal tract in a subject comprising the step of administering to the subject a composition comprising immunoglobulin G, immunoglobulin A or a combination thereof. Specifically, the subject suffers from an immune-mediated disease, for example, but not limited to arthritis, inflammatory bowel disease, skin diseases, endocrinopathies, neurodegenerative diseases and vascular diseases. In specific embodiments, supplementing the mucosal immune system comprises increasing the presence of natural antibodies in the digestive tract.

The foregoing has outlined rather broadly the features and technical advantages of the present invention in order that the detailed description of the invention that follows can be better understood. Additional features and advantages of the invention will be described hereinafter which form the subject of the claims of the invention. It should be appreciated by those skilled in the art that the conception and specific embodiment disclosed can be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description.

### BRIEF DESCRIPTION OF THE DRAWINGS

For a more complete understanding of the present invention, reference is now made to the following descriptions taken in conjunction with the accompanying drawing.

FIG. 1 shows the GI severity score after administration of oral IgG.

### DETAILED DESCRIPTION OF THE INVENTION

It is readily apparent to one skilled in the art that various embodiments and modifications can be made to the invention disclosed in this application without departing from the scope and spirit of the invention.

### A. Definitions

As used herein, the use of the word “a” or “an” when used in conjunction with the term “comprising” in the claims and/or the specification may mean “one,” but it is also consistent with the meaning of “one or more,” “at least one,” and “one or more than one.”

The term “alimentary route” as used herein is defined as any route that pertains to the digestive tube from the mouth to the anus of the subject. For example, the alimentary route includes, but is not limited to the mouth or buccal cavity, pharynx, esophagus, stomach, small intestine, large intestine or rectum. Exemplary alimentary routes of administration of drugs and/or compositions include, but are not limited to oral, rectal, sublingual or buccal.

The term “antibody” as used herein is defined as a serum immunoglobulin that has specific binding sites to combine with antigens. All antibodies have the same overall structure and are known collectively as immunoglobulins. Thus, as used herein, the terms “antibody” and “immunoglobulin” are interchangeable.

The term “autistic spectrum disorder” or “ASD”, as used herein is defined as a complex developmental disorder diagnosed on the basis of clinical characteristics (Diagnostic and Statistical Manual, 4th Edition, 1994, American Psychiatric Association). However, their condition may be influenced by various environmental factors.

The term “gammaglobulin” as used herein is defined as the protein fraction of blood serum or antiserum that contains antibodies or immunoglobulins. It is well known in the art that antisera contain heterogeneous collections of antibodies or immunoglobulins. Thus, “gammaglobulin” contains IgA, IgG, IgD, IgM and/or IgE.

The term “immune-mediated disease” as used herein refers to chronic inflammatory diseases perpetuated by antibodies and cellular immunity. Immune-mediated diseases include, for example, but not limited to, arthritis (e.g., rheumatoid arthritis and psoriatic arthritis), inflammatory bowel diseases (e.g., ulcerative colitis and Crohn's disease), endocrinopathies (e.g., type 1 diabetes and Graves disease), neurodegenerative diseases (e.g., multiple sclerosis, autistic spectrum disorder, Alzheimer's disease, Guillain-Barre syndrome, obsessive-compulsive disorder, optic neuritis, retinal degeneration, amyotrophic lateral sclerosis (ALS), Parkinson's disease, Huntington's Disease, Guillain-Barre syndrome, myasthenia gravis, and chronic idiopathic demyelinating disease (CID)), vascular diseases (e.g., autoimmune hearing loss, systemic vasculitis, and atherosclerosis), and skin diseases (e.g., dermatomyositis, systemic lupus erthematosus, discoid lupus erthematosus, scleroderma, and vasculitics).

The term “immunoglobulin” or “Ig”, as used herein is defined as a class of plasma proteins, which functions as antibodies. Immunoglobulins include IgA, IgG, IgM, IgE, or IgD and/or their subtypes, for example IgG₁, IgG₂, IgG₃, IgG₄, IgA₁, or IgA₂. IgA functions as the primary antibody that is present in body secretions, such as saliva, tears, breast milk, gastrointestinal secretions and mucus secretions of the respiratory and genitourinary tracts. IgG functions as the most common circulating antibody.

The term “in conjunction with” as used herein refers to before or prior, substantially simultaneously with or after oral administration of an antacid. Of course, the administration of a composition such as, for example, immunoglobulin, can not precede or follow administration of an antacid by so long an interval of time that the relevant effects of the substance administered first have expired. Thus, the immunoglobulin composition is usually administered within a therapeutically effective time.

The term “oral administration” as used herein includes oral, buccal, enteral or intragastric administration.

The term “pharmaceutically acceptable carrier” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The term “subject” as used herein, is taken to mean any mammalian subject to which an immunoglobulin composition is orally administered according to the methods described herein. In a specific embodiment, the methods of the present invention are employed to treat a human subject. Another embodiment includes treating a human child.

The term “therapeutically effective time” as used herein refers to a time frame in which the immunoglobulin composition and/or antacid is still active within the subject.

The term “therapeutically effective amount” as used herein refers to an amount that results in an improvement or remediation of the symptoms of the disease or condition.

The term “treating” and “treatment” as used herein refers to administering to a subject a therapeutically effective amount of an immunoglobulin composition so that the subject has an improvement in the immune-mediated neurodegenerative disease. The improvement is any improvement or remediation of the symptoms. The improvement is an observable or measurable improvement. Thus, one of skill in the art realizes that a treatment may improve the disease condition, but may not be a complete cure for the disease.

### B. Preparation of Immunoglobulin Compositions

In embodiments of the present invention, immunoglobulin compositions are administered to a subject via an alimentary route. Specifically, the immunoglobulin compositions disclosed herein may be administered orally, buccally, rectally, or sublingually. Yet further, it is envisioned that the immunoglobulin composition of the present invention can be administered via inhalation.

An immunoglobulin preparation suitable for practicing the present invention may contain varying amounts of IgA, IgG, IgM, IgE, or IgD and/or their subtypes (e.g., IgG₁, IgG₂, IgG₃, IgG₄, IgA₁ or IgA₂). In a specific embodiment of the present invention, the immunoglobulin composition is made up of predominantly IgG, IgA or a combination of IgG and IgA immunoglobulins. Yet further, the immunoglobulin composition may comprise a specific subtype of IgG or IgA or a combination thereof. Exemplary subtypes of IgG or IgA include, but are not limited to IgG₁, IgG₂, IgG₃, IgG₄, IgA₁ or IgA₂. More preferably, the immunoglobulin is a human immunoglobulin.

Yet further, it is also contemplated, that fragments of immunoglobulins are also suitable for practicing the methods of the present invention. Fragments of immunoglobulins include, but are not limited to portions of intact immunoglobulins such as Fc, Fab, Fab′, F(ab′)₂ and single chain immunoglobulins.

The immunoglobulins used according to the present invention can be obtained through isolation and purification from natural sources, for example, but not limited to blood and body secretions, such as saliva, tears, breast milk, gastrointestinal secretions and mucus secretions of the respiratory and genitourinary tracts. In other embodiments, the immunoglobulins are produced recombinatly using genetic engineering techniques well known and used in the art, such as recombinant expression or direct production in genetically altered animals, or chemical synthesis.

Isolation of native immunoglobulins for alimentary administration are prepared from blood by employing the procedures that are used in preparing immunoglobulins for parenteral administration, e.g., immunoglobulins prepared for intravenous administration (also called IVIG). Normally, blood is collected and pooled from a number of healthy volunteers. The number of blood donors is at least about 5 or 10; preferably, at least about 100; more preferably, at least about 1000; yet more preferably, at least about 10,000.

Immunoglobulins are isolated from the pooled human blood by a number of well-known methods. Such methods include, but are not limited to Cohn's alcohol fractionation (Cohn et al., 1946; Oncley et al., 1949), fractionation (Schneider et al., 1976), ultracentrifugation (Barundern et al., 1962), or the method of Kistler and Nitschmann (1962), polyelectrolyte affinity adsorption, large scale electrophoresis, ion exchange adsorption, and polyethylene glycol fractionation. Any method which fractionates immunoglobulins from a human source is used to obtain immunoglobulins suitable for use in practicing the methods of the present invention.

Immunoglobulins fractionated from pooled human blood contain predominantly IgG, smaller amounts of IgA, and yet smaller or trace amounts of IgM, IgE, IgD, with a diverse spectrum of antibody specificities and subclass distribution characteristic of the donor population. Such a preparation can also contain trace amounts of soluble CD4, CD8, and HLA molecules and certain cytokines from the plasma e.g. TGF-β. Additional preparative steps are used to enrich a particular class of immunoglobulin. For example, protein G sepharose treatment leads to an IgA predominant preparation (Leibl et al., 1996). In addition, conventional methods are employed for producing fragments of immunoglobulins. Such methods are taught by, e.g., Coligan et al., Current Protocols in Immunology, John Wiley & Sons Inc., New York, N.Y. (1994).

Further preparative steps are used in order to render an immunoglobulin preparation safe for use in the methods of the present invention. Such steps are the same as those for rendering IVIG safe, which include, but are not limited to, enzymatic modification (Fahey et al., 1963; Kneapler et al., 1977), chemical modification (Stephan, 1975; Masuko et al., 1977), reduction and alkylation (U.S. Pat. No. 3,903,262), sulfonation, structural modification (Barundern et al., 1975), treatment with β-propiolactone, treatment at low pH (Barandun et al., 1962; Koblet et al., 1976), purification by ion exchange chromatography, treatment with solvent/detergent, nanofiltration, pasteurization and sterilization. Descriptions of these methods are also be found in, e.g., Romer et al., 1982; Romer et al., 1990; and Rutter, 1994.

A commercial source of immunoglobulin appropriate for use in the methods of the present invention is Sandoglobulin I.V.® (Sandoz Pharmaceuticals), which contains 96% IgG with traces of IgA and IgM. Another commercial source of immunoglobulin is IgAbulin® (Immuno AG, Vienna, Austria), which contains predominantly IgA. Yet, an additional source is Panglobulin® IVIG, which contains primarily IgG, a small amount of IgA and traces of IgM. Yet further, another commercial source is Oralgam.

The safety standards of an immunoglobulin composition for oral administration are the same as those proposed for IVIG. For example, standards for the preparation of IVIG were proposed in 1989 in a World Health Organization (WHO) bulletin and updated in 1989 to increase the safety of prepared immunoglobulins and other blood products. Safety tests which are performed may include, e.g., sterility test, Pyrogen test, Hepatitis B antigen test, anticomplementary activity test and the like. See, e.g., A. Gardi (1984).

It is contemplated that immunoglobulins isolated from pooled human blood are made into powders by conventional freeze-drying (or lyophilization) procedure. One or more stabilizing substances are added to the immunoglobulin preparation prior to the freeze-drying process. A variety of stabilizing substances are employed including, e.g., amino acids such as glycine and lysine, carbohydrates such as dextrose, mannose, galactose, fructose, lactose, sucrose, maltose, sorbitol, mannitol and the like.

An immunoglobulin preparation in lyophilized form for use in practicing the methods of the present invention are also obtained through commercial sources. Such sources include, but are not limited to: Gammagard S/D® (Baxter Healthcare), Sandoglobulin I.V.® (Sandoz Pharmaceuticals), Polygam S/D® (American Red Cross), Venoglobulin®-I (Alpha Therapeutic), VZIG® (American Red Cross), IgAbulin® (Immuno AG, Vienna, Austria) and Intraglobin-F® (Biotest Pharma GmbH, Frankfurt, Germany).

### C. Pharmaceutical Compositions

Further in accordance with the present invention, the immunoglobulin preparation or composition suitable for oral administration is provided in a pharmaceutically acceptable carrier with or without an inert diluent. The carrier should be assimilable and edible and includes liquid, semi-solid, e.g., pastes, or solid carriers. Except insofar as any conventional media, agent, diluent or carrier is detrimental to the recipient or to the therapeutic effectiveness of an immunoglobulin preparation contained therein, its use in an administrable immunoglobulin for use in practicing the methods of the present invention is appropriate. Examples of carriers or diluents include fats, oils, water, saline solutions, lipids, liposomes, resins, binders, fillers and the like, or combinations thereof.

In accordance with the present invention, the immunoglobulin composition is combined with the carrier in any convenient and practical manner, e.g., by solution, suspension, emulsification, admixture, encapsulation, absorption and the like. Such procedures are routine for those skilled in the art.

In a specific embodiment of the present invention, an immunoglobulin composition in powder form is combined or mixed thoroughly with a semi-solid or solid carrier. The mixing can be carried out in any convenient manner such as grinding. Stabilizing agents can be also added in the mixing process in order to protect the immunoglobulin composition from loss of therapeutic activity through, e.g., denaturation in the stomach. Examples of stabilizers for use in an orally administrable immunoglobulin preparation include buffers, antagonists to the secretion of stomach acids, amino acids such as glycine and lysine, carbohydrates such as dextrose, mannose, galactose, fructose, lactose, sucrose, maltose, sorbitol, mannitol, etc., proteolytic enzyme inhibitors, and the like.

Further, an immunoglobulin composition which is combined with a semi-solid or solid carrier can be further formulated into hard or soft shell gelatin capsules, tablets, or pills. More preferably, gelatin capsules, tablets, or pills are enterically coated. Enteric coatings prevent denaturation of the immunoglobulin composition in the stomach or upper bowel where the pH is acidic. See, e.g., U.S. Pat. No. 5,629,001. Upon reaching the small intestines, the basic pH therein dissolves the coating and permits the immunoglobulin composition to be released and absorbed by specialized cells, e.g., epithelial enterocytes and Peyer's patch M cells.

In a specific embodiment of the present invention, Panglobulin® is encapsulated in a gelatin capsule (IgPO, encapsulated Panglobulin ®).

In another embodiment, a powdered immunoglobulin composition is combined with a liquid carrier such as, e.g., water or a saline solution, with or without a stabilizing agent. Such preparations reconstituted in solutions are also obtained through commercial sources. Such commercial sources include BayRho-D® Full Dose (Bayer Biological), BahRho-D® Mini-Dose (Bayer Biological), Gamimune N®, 5% (Bayer Biological), Gamimune N®, 5% Solvent/Detergent Treated (Bayer Biological), Gamimune NO, 10% (Bayer Biological), Gamimmune N 5% (Miles), Gammagard S/D® (Baxter Healthcare), Isiven V.I. 2.5% (Isiven), MICRhoGAM® (Ortho Diagnostic), RhoGAM® (Ortho Diagnostic), Sandoglobulin I.V.® (Sandoz Pharmaceuticals), Polygam S/D® (American Red Cross), Venoglobulin-S® 5% Solution Solvent Detergent Treated (Alpha Therapeutic), Venoglobulin-S® 10% Solution Solvent Detergent Treated (Alpha Therapeutic), and IgAbulin® (Immuno AG, Vienna, Austria).

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In other embodiments, one may use eye drops, nasal solutions or sprays, aerosols or inhalants in the present invention. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation.

Upon formulation, solutions are administered in a manner compatible with the dosage formulation and in such amount as is therapeutically effective to result in an improvement or remediation of signs and/or symptoms. The formulations are easily administered in a variety of dosage forms such as ingestible solutions, drug release capsules and the like. Some variation in dosage can occur depending on the condition of the subject being treated. The person responsible for administration can, in any event, determine the appropriate dose for the individual subject. Moreover, for human administration, preparations meet sterility, pyrogenicity, general safety and purity standards as required by FDA Office of Biologics standards.

### D. Treatment Using Oral Immunoglobulin

According to the present invention, a subject that is suspected of an immune-mediated disease or a subject suffering from an immune-mediated disease is treated with the immunoglobulin composition of the present invention. The treatment comprises administering via an alimentary route to a subject a therapeutically effective amount of an immunoglobulin composition so that the subject has an improvement in the immune-mediated disease.

Immune-mediated diseases of the present invention include, for example, but are not limited to, arthritis (e.g., rheumatoid arthritis and psoriatic arthritis), inflammatory bowel diseases (e.g., ulcerative colitis and Crohn's disease), endocrinopathies (e.g., type 1 diabetes and Graves disease), neurodegenerative diseases (e.g., multiple sclerosis, autistic spectrum disorder, Alzheimer's disease, Guillain-Barre syndrome, obsessive-compulsive disorder, optic neuritis, retinal degeneration, amyotrophic lateral sclerosis (ALS), Parkinson's disease, Huntington's Disease, Guillain-Barre syndrome, myasthenia gravis, and chronic idiopathic demyelinating disease (CID)), vascular diseases (e.g., autoimmune hearing loss, systemic vasculitis, and atherosclerosis), and skin diseases (e.g., dermatomyositis, systemic lupus erthematosus, discoid lupus erthematosus, scleroderma, and vasculitics).

In specific embodiments, a subject suspected of an immune-mediated neurodegenerative diseases, such as autistic spectrum disorder (ASD), or a subject suffering from an immune-mediated disease neurodegenerative diseases, such as autistic spectrum disorder (ASD), is treated with the immunoglobulin composition of the present invention. The treatment comprises administering via an alimentary route to a subject a therapeutically effective amount of an immunoglobulin composition so that the subject has an improvement in the immune-mediated disease neurodegenerative diseases, such as autistic spectrum disorder (ASD). Yet further, it is also envisioned that the treatment of neurodegenerative diseases also includes treatment of the associated GI manifestations.

The improvement is any remediation of the symptoms associated with the disease or condition. The therapeutic effects of an immunoglobulin composition are believed to result from a blockade of Fc-Receptors (Samuelsson et al., 2000), a neutralization of an autoantibody by anti-idiotype antibodies present in the immunoglobulin composition, binding and down-regulation by anti-idiotype antibodies of the B-cell receptor for an antigen thereby decreasing the autoantibody production (Dietrich et al., 1993), attenuation of complement mediated and immune complex-mediated tissue damage, increasing production of anti-inflammatory cytokine (Prasad et al., 1998), suppression of proliferation of antigen-specific T-cells (Aktas et al., 2001), induction of apoptosis of activated T and B cells (Prasad et al., 1998); neutralization of microbial toxins; or a combination thereof.

1. Administration of Immunoglobulin

In accordance with the present invention, an immunoglobulin composition provided in any of the above-described pharmaceutical carriers is administered via an alimentary route to a subject suspected of or having an immune mediated disease. The precise therapeutically effective amount of immunoglobulin composition to be administered is determined by a physician with consideration of individual differences in age, weight, disease severity and response to the therapy. Alimentary routes of administration include, but are not limited to oral, nasal, buccal, sublingual or rectal. More preferably, the alimentary route is oral. Oral administration of the immunoglobulin composition includes oral, buccal, enteral or intragastric administration. It is also envisioned that the composition is a food additive. For example, the composition is sprinkled on food or added to a liquid prior to ingestion.

In a specific embodiment, an immunoglobulin composition is administered about one to five times a day at a dose of about 100 mg/day-1000 mg/day. The immunoglobulin composition is administered before, during, or after a meal. In a further embodiment, an immunoglobulin composition is administered once a day at a dose of 100-1000 mg/day. Daily administration usually occurs before bedtime.

To further reduce the degree of inactivation of an immunoglobulin composition in the stomach of an individual undergoing treatment according to the methods of the present invention, an antacid is administered just prior or immediately after oral administration of the immunoglobulin composition. An antacid is also given simultaneously with the immunoglobulin composition. Examples of appropriate antacids include, but are not limited to sodium bicarbonate, magnesium oxide, magnesium hydroxide, calcium carbonate, magnesium trisilicate, magnesium carbonate, and aluminum hydroxide gel.

In specific embodiments, the antacid is aluminum hydroxide or magnesium hydroxide such as Maalox® or Mylanta®, which are commercially available. In further specific embodiments, the antacid is an H₂ blocker such as Cimetidine or Ranitidine. The dose ranges are between 15 ml and 30 ml for Mylanta, and between 400 and 800 mg per day for Cimetidine.

In accordance with the present invention, the time needed to complete a course of the treatment is determined by a physician and may range from as short as one day to more than one week. A preferred course of treatment is from 2 to 8 weeks. More preferably, the course of treatment lasts for eight weeks. A course of treatment is repeated as often as necessary, as determined by a physician, in order to maintain or extend the therapeutic benefit to the patient.

2. Determining Improvement

After the immunoglobulin composition is administered to a subject, the subject is evaluated to determine if the treatment results in an improvement of the subject. The improvement is any improvement or remediation of signs or symptoms of the disease or condition.

The improvement is an observable improvement, such as gastrointestinal signs or symptoms, social interaction, communication, and/or behavior. Such improvements are measured using evaluation systems, which are well known and used in the clinical field. It is also contemplated that the improvement is a measurable improvement, for example, immunological parameters.

In specific embodiments, an ASD subject that is treated according to the methods described herein is evaluated using standard evaluation systems to determine an observable improvement, for example, evaluation of aberrant behavior (e.g., Aberrant Behavior Check List (Aman et al., 1997); Childhood Autism Rating Scale (Coniglio et al., 2001); evaluation of child development (e.g., Clinical Global Impression (Sandler et al., 1999), Preschool Language Scale (Dunn-Geier et al., 2000)); or QTL assessment for children (Collier et al., 2000). Other clinical symptoms that are observed for ASD include, but are not limited to frequency of bowel movement, stool consistency, color and smells of stools, etc. Yet further, immunological parameters are also measured using standard techniques known in the art. These parameters include, but are not limited to cytokine production in response to common dietary antigens (gliadin, casein, α-lactoalbumin, and β-lactoglobulin), LPS, and MBP, IFN-γ, TNF-α, IL-5, IL-1βIL-6, IL-10, sTNFRII, and IL-12p40, and markers for the gastrointestinal tract inflammation (e.g., calprotectin levels in the stool).

3. Immune Replacement Therapy

Selective immunodeficiency in the gastrointestinal tract determines the predisposition to many autoimmune diseases. In view of this, immune deficiency in the gastrointestinal is correctable by immune replacement therapy. Human immunolobulin contains a full spectrum of antibodies to microbes, including those that are absent in genetically predisposed patients who develop autoimmune disease. Since immunoglobulin administered intravenously is poorly secreted into the gastrointestinal tract, it is necessary to give immunoglobulin via an alimentary route, such as an oral route. Thus, the majority of immunoglobulin given via an alimentary route (i.e., orally) remains intact in the gastrointestinal tract and can neutralize bacteria, viruses, fungi and parasites and their products.

It is envisioned that immunoglobulins that are administered via an alimentary route are absorbed and processed by specialized cells in the mucosa tissues of the digestive tract, e.g., epithelial enterocytes and Peyer's patch M cells in the gut-associated lymphoid tissue, which permits the establishment of self-tolerance and inhibition of autoimmune reactions in the subjects. Yet further, administered immunoglobulins can bind intestinal immunoglobulin G receptors, i.e., FcγR, and such binding initiate modulation of autoimmune disorders, that is, Fc-mediated immunomodulation. This Fc-mediated immunomodulation can occur via immunoglobulin binding of, but not limited to, the intestinal FcγRBP, i.e., Fcγ Binding Protein (Harada et al., 1991), which plays an important role in inflammation and immunomodulation in humans (Harada et al., 1997) with autoimmune diseases (Kobayashi et al., 2001); the IgG receptor FcRn in the intestinal epithelium (Israel et al., 1997) which prevents IgG catabolism (Junghans and Anderson, 1996) and thus, has a role in modulating the increased serum immunoglobulin of some autoimmune disorders (Bleeker et al., 2001).

Thus, the present invention contemplates administering immunoglobulins via an alimentary route to stimulate, enhance or supplement the mucosal immune system and modulate autoimmune disorders resulting in a treatment for immune-mediated diseases.

4. Combination Treatments

It is also well within the scope of the present invention to administer immunoglobulin compositions in combination with a known treatment for the immune-mediated disease that is being treated. For example, oral immunoglobulin is administered to an ASD patient in combination with a therapy for dietary protein intolerance or a behavioral therapy program. It is well within the knowledge of those of skill in the art to determine the appropriate therapies to use in combination with the oral immunoglobulin therapy.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Oral Treatment

Subjects are administered the immunoglobulin composition orally once a day before bedtime, as an add-on to their background therapy for ASD and dietary protein intolerance.

Subjects receive capsules containing IgG and IgA once daily for 8 weeks (420 mg/day).

After 8 weeks, the following parameters are evaluated and the results are compared to those obtained prior to treatment. Immunological parameters are measured, for example, cytokine production in response to common dietary antigens (gliadin, casein, α-lactoalbumin, and β-lactoglobulin), LPS, and MBP. Cytokine levels are determined at protein and mRNA levels.

Other parameters that are measured include IFN-γ, TNF-α, IL-5, IL-1β, IL-6, IL-10, sTNFRII, and IL-12p40. Markers for the GI tract inflammation are measured, such as calprotectin levels in the stool. Clinical symptoms, such as frequency of bowel movement, stool consistency, color and smells of stools, etc. are documented.

Other evaluations include, evaluation of aberrant behavior, (Aman et al., 1997), Childhood Autism Rating Scale (Coniglio et al., 2001), Evaluation of Child Development: (Neuropsychiatric assessment), clinical Global Impression (Sandler et al., 1999), Preschool Language Scale (Dunn-Geier et al., 2000), QTL assessment for children (Collier et al., 2000)

Aberrant innate immune responses in the GI tract make children with ASD more vulnerable to sensitization to common dietary proteins (DP), resulting in chronic GI inflammation and even autoimmune condition. Thus, the present invention envisions that oral administration of immunoglobulins prevents or attenuates GI inflammation induced by dietary protein and intestinal microbes by exerting various anti-inflammatory and immunomodulating actions.

Example 2

### Oral Treatment with Oralgam™

Subjects were administered the immunoglobulin composition orally once a day before bedtime, as an add-on to their background therapy for ASD and dietary protein intolerance.

Subjects received a single oral dose of human IgG, prior to bedtime, for 8 weeks. The single dose consisted of three 140mg capsules of IgG.

Clinical assessments were carried out at prior to treatment (baseline), 4 weeks, 8 weeks, and 12 weeks. Each assessment consisted of a physical examination and an assessment of clinical activity (GI severity score, Physician global assessment, Patient global assessment, and Autism Behavior Checklist). Clinical symptoms, such as frequency of bowel movement, stool consistency, color and smells of stools, etc. were documented.

Laboratory testing consisted of urinalysis, cell blood count (CBC) with manual differential, Westergren erythrocyte sedimentation rate (ESR), C reactive protein (CRP), chemistry 14 panel, Quantitative Immunoglobulins (QIGs), a stool culture for Clostridium difficile, and freezing of a serum aliquot. After 8 weeks, the same parameters were evaluated and the results were compared to those obtained prior to treatment.

A clinical response was defined as a drop in the GI severity index score of at least 4 points from baseline. A clinical remission was defined by a GI Severity score of ≦4 with an overall improvement of at least 4 points from baseline.

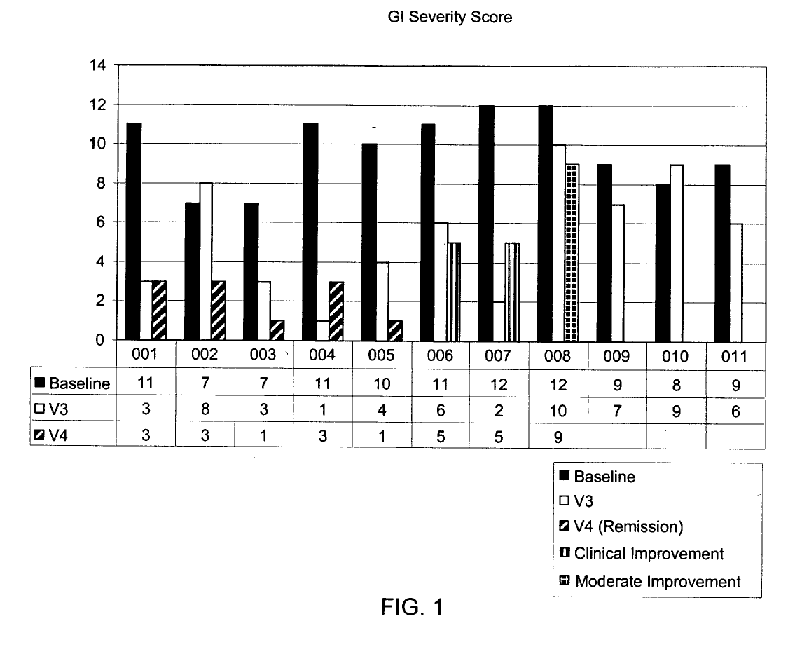

Table 1 and FIG. 1 show that 55% of the patients had remission of GI symptoms and 78% of the patents had improvement of GI signs and symptoms. Yet further, Table 2 shows that there was a 21.8 point decrease in the ABC average score, which suggested an improvement in behavior.

TABLE 1
CLINICAL RESULTS AFTER 8 WEEKS

| Patient | GI Severity | ABC | Phys Global | Parent Global | Gender | Visit |
|---|---|---|---|---|---|---|
| 002 | 11 | 129 | Severe | Severe | Male - 7 | Baseline |
| | 3 | 111 | No change | No change | | 30 days |
| | 3 | 111 | No change | No change | | 60 days |
| 003 | 12 | 101 | Severe | Moderate | Male - 4 | |
| | 10 | 101 | Min. improve | Min. improve | | |
| | 9 | 75 | Much impr. | Much impr. | | |
| 004 | 11 | 94 | Mild | Moderate | Male -4 | |
| | 6 | 94 | Much impr. | Much impr. | | |
| | 5 | 72 | Much impr. | Much impr. | | |
| 005 | 9 | 86 | Moderate | Moderate | Male - 7 | |
| | 7 | 86 | Min. improve | Much impr. | Vomiting - discontinued | |
| 006 | 7 | 85 | Severe | Severe | Male - 3 | |
| | 8 | 90 | Much worse | Much worse | | |
| | 3 | 91 | No change | No change | | |
| 007 | 8 | 76 | Mild | Moderate | Male - 4 | |
| | 9 | 70 | Min. improve | Min. improve | | |
| | | 60 | Min. improve | Min. improve | | |
| 008 | 7 | 94 | Moderate | Moderate | Male - 7 | |
| | 3 | 63 | Min. improve | Min. improve | | |
| | 1 | 43 | Min. improve | Min. improve | | |
| 009 | 9 | 110 | Moderate | Moderate | Male - 3 | |
| | | | | | Anorexic, | |
| | 6 | 110 | No change | No change | discontinued drug | |
| 010 | 12 | 117 | Moderate | Moderate | Male - 3 | |
| | 2 | 96 | Much impr. | Much impr. | | |
| | 5 | 96 | Much impr. | Very much impr. | | |
| 011 | 11 | 79 | Mild | Mild | Male - 3 | |
| | 1 | 61 | Min. improve | Min. improve | | |
| | 3 | 33 | Much impr. | Much impr. | | |
| 012 | 10 | 80 | Severe | Moderate | Male - 7 | |
| | 4 | 85 | Much worse | Much worse | | |
| | 1 | 82 | No change | No change | | |
| 013 | 7 | 45 | Moderate | Moderate | Male - 7 | |
| | | | | | Rash - discontinued | |

TABLE 2
AVERAGE ABC

| ABC | Average Score |
|---|---|
| Baseline | 95.5 |
| 4 weeks | 87.9 |
| 8 weeks | 73.7 |

Thus, the data suggested that oral administration of immunoglobulins prevented or attenuated GI inflammation or GI signs and symptoms and improved the overall behavior as determined by the Autism Behavior Checklist (ABC).

### Example 3

### The use of Oral Gammaglobulin for the Treatment of Autism and Related Gastrointestinal Problems

A subject was diagnosed as “autistic” at the age of 2.5 years. At that time, a MRI or CNS showed localized demyelination. The subject also suffered an additional major regression following a VZV vaccination at age 3.75 years. An MRI following this episode of major regression showed increased demyelination. An elimination diet, secretin, numerous supplements and behavioral therapy have been tried with limited responses.

Further testing indicated autoimmune/inflammatory condition in the cerebral spinal fluid (CSF). Notably, the subject was positive for anti-MBP antibodies in serum and CSF. The subject was also noted to have elevated serum levels of proinflammatory (IL-1β, IL-6, IL-12p40, IL-18 and TNF-α) and counter-regulatory cytokines (IL-1ra, TGF-β, sTNFRI, and sTNFRII levels in the serum). More importantly, these cytokine levels were elevated in CSF as like seen in MS patients, indicating autoimmune conditions in the CNS. STNFRI and sTNFRII were notably elevated.

For treatment, the patient was administered 400 mg of an immunoglobulin composition daily in the form of Panglobulin (IVIg) dissolved in about 10 cc of water and taken orally at bedtime.

After 6-7 weeks of treatment, the GI problems improved as well as his cognitive speech.

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

U.S. Pat. No. 3,903,262

Aktas O., et al., J. Neuroimmunol. 114:160-167, 2001.

Aman MG., et al., 1997. Am. J. Mental Retard. 101:521-534.

Barandun et al., Vox Sang. 7: 157-174, 1962;

Barundern et al., Mong. Allergy 9: 39-60, 1975

Barundern et al., Vox Sang. 7: 157-174, 1962

Bendtzen K. et al., Immunol. Today 19: 209-211.

Berney T P. et al., 2000. British Journal of Psychiatry. 176:20-25.

Bleeker et al., 2001. Blood 98(10): 3136-42.

Casswall et al., 1996. Acta Paediatr 85(9): 1126-8.

Cohn et al., J. Am. Chem. Soc. 68:459-475,1946;

Coligan et al., Current Protocols in Immunology, John Wiley & Sons Inc., New York, N.Y. (1994).

Coniglio S. J. et al., 2001. J. Ped. 138: 649-655.

Dietrich G. et al., 1993. Eur. J. Immunol. 23: 2945-2950.

Dunn-Geier J. et al., 2000. Develop Med Child Neurol 42:796-802.

Fahey et al., J. Exper. Med.,118: 845-868, 1963;

Furlano R. et al., 2001. J. Pediatr. 138: 366-372.

Ghetie, V. and E. S. Ward 2000. Annu Rev Immunol 18: 739-66.

Greenberg, P. D. et. al. 1996. J Acquir Immune Defic Syndr Hum Retrov 13(4): 348-54.

Gupta, S. et al., 1996. J. Autism Develop. Dis. 26:439-452.

Harada et al., 1991. Immunology 74(2): 298-303.

Harada et al., 1997. J Biol Chem 272(24): 15232-41.

Israel et al., 1997. Immunology 92(1): 69-74.

Junghans, R. P. and C. L. Anderson (1996). Proc Natl Acad Sci U S A 93(11): 5512-6.

Jyonouchi et al., 2001. FASEB J. 15: A939 (Abst. 726.12).

Kazatchkine and Kaveri, N Engl J Med., 345(10):747-55, 2001.

Kistler and Nitschmann Vox Sang 7: 414-424, 1962.

Kneapler et al., Vox Sang.32: 159-164, 1977.

Koblet et al., Vox Sang.31: 141-151, 1976

Krause et al., 2000. Crit Rev Immunol. 20:1-16.

Leibl et al., J. Chromatogr B. Biomed. Appl. 678(2):173-180 (1996)).

Lord et al., 2000. Autism Dev Disord Jun;30(3):205-23

Masuko et al., Vox Sang. 32: 175-181, 1977

Meyer et al., 2000. Joint, Bone, Spine: Revue du Rhumatisme. 67: 384-392.

Oncley et al., J. Am. Chem. Soc., 71: 541-550, 1949

Prasad et al., 1998. J. Immunol. 161: 3781-3790.

Romer et al., Vox Sang. 42: 62-73, 1982;

Romer et al., Vox Sang. 42: 74-80, 1990;

Rutter, J. Neurosurg. Psychiat. 57 (Suppl.): 2-5, 1994

Samuelsson et al., 2001. Science. 291:484-486, 2001.

Sandier et al., 1999. N Engl J Med. 341:1801-1806.

Schneider et al., Vox Sang.31: 141-151, 1976

Singh et al., 1993. Behavior, Immun. 7:97-103.

Singh et al., 1996. J. Neuroimmunol. 66:143-145.

Singh et al., 1997. Biol. Psychiatry 31: 753-755.

Stephan, Vox Sang. 28: 422-437, 1975;

Tiwana et al., 1999. Infect Immun. 67: 2769-2775.

Wakefield et al., 2000. Am. J. Gastroenterol. 95:2285-95.

Xu et al., 1998. Am. J. Pathol. 153:1257-1266.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein can be utilized according to the present invention. Accordingly, the appended claims are intended to include within their scope such processes, machines, manufacture, compositions of matter, means, methods, or steps.

We claim:

1. A method of treating an immune-mediated neurodegenerative disease in a subject comprising the step of administering to said subject via an alimentary route an immunoglobulin composition in an amount sufficient to provide an improvement in the neurodegenerative disease in said subject.

2. The method of claim 1, wherein said alimentary route is selected from the group consisting of oral, rectal, sublingual and buccal.

3. The method of claim 2, wherein said alimentary route is oral.

4. The method of claim 1, wherein said neurodegenerative disease is selected from the group consisting of multiple sclerosis, autism and Alzheimer's disease.

5. The method of claim 1, wherein said immunoglobulin composition comprises a native immunoglobulin or a recombinant immunoglobulin.

6. The method of claim 1, wherein said immunoglobulin composition comprises human immunoglobulin.

7. The method of claim 5, wherein said human immunoglobulin composition comprises human immunoglobulin G.

8. The method of claim 5, wherein said human immunoglobulin composition comprises human immunoglobulin G and immunoglobulin A.

9. The method of claim 5, wherein said human immunoglobulin composition comprises human immunoglobulin A.

10. The method of claim 1, wherein said immunoglobulin composition is dispersed in a pharmaceutically acceptable carrier.

11. The method of claim 3 further comprises administering an antacid in conjunction with said immunoglobulin composition.

12. A method of treating an autistic spectrum disorder in a subject comprising the step of orally administering to said subject an immunoglobulin composition in an amount sufficient to provide an improvement in the autistic spectrum disorder in said subject.

13. The method of claim 12, wherein said immunoglobulin composition comprises human immunoglobulin.

14. The method of claim 13, wherein said human immunoglobulin composition comprises human immunoglobulin G.

15. The method of claim 13, wherein said human immunoglobulin composition comprises human immunoglobulin G and immunoglobulin A.

16. The method of claim 13, wherein said human immunoglobulin composition comprises human immunoglobulin A.

17. The method of claim 12, wherein said immunoglobulin composition is dispersed in a pharmaceutically acceptable carrier.

18. The method of claim 12 further comprising administering an antacid in conjunction with said immunoglobulin composition.

19. The method of claim 18, wherein said antacid is administered prior to said immunoglobulin composition.

20. The method of claim 18, wherein said antacid is administered simultaneously with said immunoglobulin composition.

21. A method of treating an immune-mediated disease comprising the step of supplementing a mucosal immune system.

22. The method of claim 21, wherein said immune-mediated disease is an autistic spectrum disorder.

23. The method of claim 22, wherein supplementing the mucosal immune system comprises increasing the amount of immunoglobulin G, immunoglobulin A or a combination thereof in the gastrointestinal tract.

24. The method of claim 23, wherein immunoglobulin G, immunoglobulin A or a combination is administered via an alimentary route.

25. The method of claim 24 said alimentary route is selected from the group consisting of oral, rectal, sublingual and buccal.

26. The method of claim 25, wherein said alimentary route is oral.

27. A method of enhancing a mucosal immune response in the gastrointestinal tract in a subject comprising the step of administering to said subject a composition comprising immunoglobulin G, immunoglobulin A or a combination thereof.

28. The method of claim 27, wherein administering is via an alimentary route selected from the group consisting of oral, rectal, sublingual and buccal.

29. The method of claim 28, wherein said alimentary route is oral.

30. The method of claim 27, wherein supplementing the mucosal immune system comprises increasing the presence of natural antibodies in the digestive tract.

31. The method of claim 27, wherein said subject suffers from an immune-mediated disease.

32. The method of claim 27, wherein said immune-mediated disease is selected from the group consisting of arthritis, inflammatory bowel disease, skin diseases endocrinopathies, neurodegenerative diseases and vascular diseases.
